(19) United States
(12) Patent Application Publication
(10) Pub. No.: US 2023/0001031 A1
(43) Pub. Date: Jan. 5, 2023

(54) DISINFECTING DEVICE

(71) Applicant: JC Technologies LLC, Auburn, AL (US)

(72) Inventors: John Edgar Curry, Auburn, AL (US); Harry Edmar Schulz, Auburn, AL (US)

(21) Appl. No.: 17/942,443

(22) Filed: Sep. 12, 2022

Related U.S. Application Data
(63) Continuation-in-part of application No. 17/467,536, filed on Sep. 7, 2021.
Application No. 17/467,536 is a continuation of application No. 17/151,562, filed on Jan. 18, 2021, now Pat. No. 11,110,189.
Application No. 17/151,562 is a continuation-in-part of application No. 16/900,243, filed on Jun. 12, 2020, now Pat. No. 10,894,101.

(51) Int. Cl. A61L 2/10; H05K 7/20

(52) U.S. Cl. CPC A61L 2/10; H05K 7/20254; H05K 7/20272; A61L 2202/26; A61L 2202/11

(57) ABSTRACT

The present disclosure provides a garment comprising: a disinfecting device comprising: a cavity, a sensor operative to actuate upon one or more objects entering the cavity, and a disinfecting portion configured to disinfect the one or more objects within the cavity, the disinfecting portion being actuated upon receiving an indication from the sensor of the one or more objects entering the cavity; and a lighting apparatus disposed on an outer surface of the garment in operative communication with the disinfecting device, the lighting apparatus comprising: a clean visual indicator, an unclean visual indicator, and a switch configured to: responsive to a predetermined amount of time passing and the disinfecting portion not being actuated, display the unclean visual indicator and stop display of the clean visual indicator, and responsive to the disinfecting portion being activated, display the clean visual indicator and stop display of the unclean visual indicator.

### RELATED APPLICATIONS

This application is a Continuation-In-Part of U.S. application Ser. No. 17/467,536 filed on Sep. 7, 2021, which is a Continuation of U.S. application Ser. No. 17/151,562 filed on Jan. 18, 2021, which issued on Sep. 7, 2021 as U.S. Pat. No. 11,110,189, which is a Continuation-In-Part of U.S. application Ser. No. 16/900,243 filed on Jun. 12, 2020, which issued on Jan. 19, 2021 as U.S. Pat. No. 10,894,101, which are incorporated herein by reference in its entirety.

It is intended that the above-referenced application may be applicable to the concepts and embodiments disclosed herein, even if such concepts and embodiments are disclosed in the referenced applications with different limitations and configurations and described using different examples and terminology.

### FIELD OF DISCLOSURE

The present disclosure relates to disinfecting surfaces, and more specifically, to disinfecting surfaces of hands.

### BACKGROUND

Disinfecting hands is one of the most effective ways to avoid spreading contagious diseases. There are many methods used, to disinfect hands such as, soap and water, waterless soap, or solvent-based hand sanitizer. Each of these methods have disadvantages. For example, the use of soap and water is done at a fixed fixture such as a sink or hand washing station, the use of solvent-based hand sanitizers may be problematic since they are quickly depleted, and may not be available under some conditions.

A quick, effective, easily portable, and convenient device for sanitizing hands is desired. This need and other needs are satisfied by the various aspects of the present disclosure.

### SUMMARY

In accordance with the purposes of the present disclosure, as embodied and broadly described herein, the present disclosure, in one aspect, relates to devices, garments, and methods for disinfecting surfaces, such as, for example, skin surfaces. In another aspect, a device of the present disclosure can include a device comprising: at least one opening, the at least one opening being configured receive an object; the inner cavity within the at least one opening, the inner cavity comprising: a light emitting diode (LED), a heat sink connecting the LED to the thermal reservoir, and an insulating layer arranged on the thermal reservoir, the insulating layer comprising an orifice arranged over the LED such that light from the LED passes through the orifice of the insulating layer; and a power source configured to power the LED.

In another aspect, the present disclosure relates to a garment comprising: a disinfecting device comprising: a cavity, a sensor operative to actuate upon one or more objects entering the cavity, and a disinfecting portion configured to disinfect the one or more objects within the cavity, the disinfecting portion being actuated upon receiving an indication from the sensor of the one or more objects entering the cavity; and a lighting apparatus disposed on an outer surface of the garment in operative communication with the disinfecting device, the lighting apparatus comprising: a clean visual indicator, an unclean visual indicator, and a switch configured to: responsive to a predetermined amount of time passing and the disinfecting portion not being actuated, display the unclean visual indicator and stop display of the clean visual indicator, and responsive to the disinfecting portion being activated, display the clean visual indicator and stop display of the unclean visual indicator.

In another aspect, the present disclosure relates to a disinfecting system comprising: a dispensing portion configured to administer a predetermined amount of sanitizing fluid, and a first proximity sensor coupled to the disinfecting portion and operative to transmit a signal responsive to the following steps being performed: a second proximity sensor being within a predetermined distance of the first proximity sensor, and administration (e.g., dispensing) of the predetermined amount of sanitizing fluid; and the lighting apparatus disposed on an outer surface of a garment of a user and in operative communication with the disinfecting device, the lighting apparatus comprising: a display portion configured to visually display one of a clean visual indicator and an unclean visual indicator, and a switch coupled to the second proximity sensor, the switch configured to: responsive to a predetermined amount of time passing and the disinfecting portion not being actuated, display the unclean visual indicator on the display portion, and responsive to receiving the signal transmitted from the first proximity sensor, display the clean visual indicator on the display portion.

In another aspect, the present disclosure relates to a disinfecting system that includes a disinfecting device comprising: a cavity, a bacteria detection sensor operative to: actuate upon one or more objects entering the cavity, and detect a bacterial amount on the object, a disinfecting portion configured to disinfect the one or more objects within the cavity, the disinfecting portion being actuated upon receiving an indication from the sensor of the one or more objects entering the cavity; and a lighting apparatus disposed on an outer surface of a garment in operative communication with the disinfecting device, the lighting apparatus comprising: a display portion, and a switch configured to: responsive to a predetermined amount of time passing and the disinfecting portion not being actuated, display the unclean visual indicator on the display portion, and responsive to the disinfecting portion being activated and the bacteria detected on the object, via the bacteria detection sensor, falling below a predetermined threshold, display the clean visual indicator on the display portion.

In another aspect, the present disclosure relates to a device comprising: at least one opening, the at least one opening being configured receive an object; an external housing configured to secure to a garment; an entry portion configured to be in a closed state and an open state; a switch disposed on the entrance portion; and an internal housing within the at least one opening, the internal housing comprising: a light emitting diode (LED), a heat sink connecting the LED to the thermal reservoir, and an insulating layer arranged on the thermal reservoir, the insulating layer comprising an orifice arranged over the LED such that light from the LED passes through the orifice of the insulating layer.

In another aspect, the present disclosure relates to device comprising: a light emitting diode (LED); a heat sink connecting the LED to a liquid circulation channel, the heat sink being configured to transfer heat from the LED to the liquid circulation channel; and an insulating layer arranged on the thermal reservoir, the insulating layer including an orifice arranged over the LED such that light from the LED passes through the orifice in the insulating layer.

In further aspects, the present disclosure also relates to garments comprising the disclosed devices.

In still further aspects, the present disclosure also relates to methods for making and using the disclosed systems, devices, and garments. Additional aspects of the present disclosure will be set forth in part in the description which follows, and in part will be obvious from the description, or can be learned by practice of the present disclosure. The advantages of the present disclosure will be realized and attained by means of the elements and combinations particularly pointed out in the appended claims. It is to be understood that both the foregoing general description and the following detailed description are exemplary and explanatory only and are not restrictive of the present disclosure, as claimed.

### BRIEF DESCRIPTION OF THE DRAWINGS

FIG. 1 illustrates an exploded planar view of a flexible disinfecting light device.

FIG. 2 illustrates a circuit.

FIG. 3 illustrates a perspective view of an example embodiment of a pocket shaped disinfectant device.

FIG. 4 illustrates a garment that includes the pocket.

FIG. 5 illustrates the thermal reservoir attached to the backing sheet.

FIG. 6 illustrates a top view of the deposition of LEDs and heatsinks on the reservoir layer.

FIG. 7 illustrates a top view of the resulting structure following the arrangement of the thermal (insulating) layer on the heat reservoir.

FIG. 8 illustrates a top view of the resulting structure following application of the top layer.

FIG. 9 illustrates a plan view of an alternate embodiment of a thermal layer.

FIG. 10 shows a switch that is attached to a flexible elastic component.

FIG. 11 illustrates the arrangement of the flexible elastic components when set in a substantially parallel position.

FIG. 12 illustrates the flexing deformation of the flexible elastic components.

FIG. 13 illustrates an embodiment of a trouser garment.

FIG. 14 illustrates a perspective view of an example embodiment of a disinfecting device.

FIG. 15 illustrates an exploded planar view of an alternate top layer.

FIG. 16 illustrates a computing device 3000.

FIG. 17 illustrates a lighting apparatus 1700.

FIG. 18 illustrates an alternate top view of the deposition of LEDs.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of dispensing fluids, embodiments of the present disclosure are not limited to use only in this context. For example, the principles and techniques disclosed herein may be understood to be applicable for adaptively dispensing other kinds of substances based on associated characteristics.

As discussed above, a portable, quick and effective system for disinfecting hands is desired. In this regard, the use of ultraviolet (UV) lights may be employed to disinfect surfaces. Indeed, UV lights have been found to be effective in disinfecting hands and other surfaces.

UV lights for disinfecting surfaces are often arranged in large fixtures or other devices. These are found in commercial or research settings for disinfecting foods and beverages, hospital rooms, or medical equipment. As discussed above, it is desirable for a system that disinfect hands while being portable and convenient.

The system and devices described herein includes a light emitting diode (LED) array that disinfects the surfaces of objects such as skin of the hands of the user. The device uses LEDs that emit ultraviolet light (and other light frequencies in some embodiments) that disinfects surfaces that it shines upon.

Previous UV disinfection devices were typically fixtures and not portable. Users would place the hands into a compartment with UV light for a period of time. Such a solution is not portable however, and is thus not convenient for most users. Described herein is an apparatus that is portable and emits ultraviolet light to disinfect the hands of the user while the user is undertaking their daily activities.

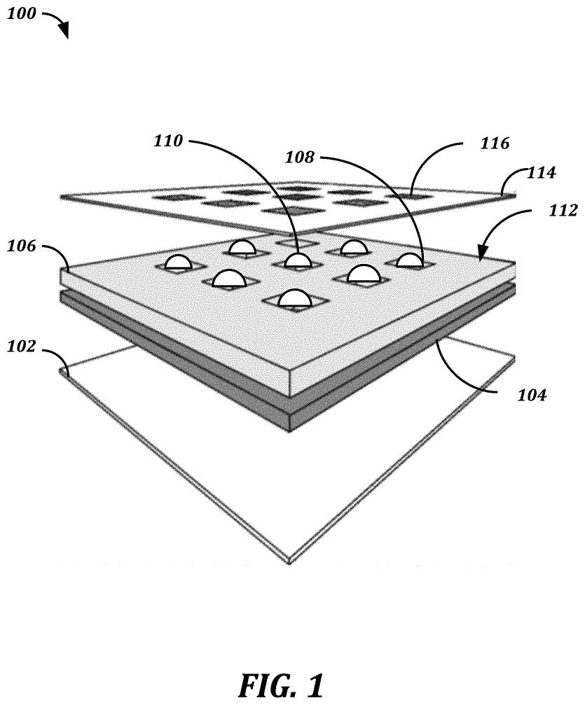

FIG. 1 illustrates an exploded planar view of a flexible disinfecting light device 100. The device 100 includes a backing cloth 102. The backing cloth 102 may include for example, any number of layers of material including, woven cloth, plastic sheet material, synthetic and natural fiber material. The backing 102 is attached to a thermal reservoir 104.

The thermal reservoir 104 may be deformable and may include any number of channels or cavities that contain a fluid that is operative to disperse heat. The thermal reservoir 104 may connect to the cloth 102 by, for example, stitching, an adhesive or welding process.

The thermal layer (insulating layer) 106 connect to thermal reservoir 104. Thermal layer 106 may include any suitable material such as, for example, neoprene or other similar materials that are flexible, deformable, and are resistant to heat. Thermal layer 106 may include a number of orifices or cavities 108. Cavities 108 may expose LEDs (light emitting devices diodes 110). LEDs 110 in the illustrated embodiment are shown as protruding from the surface 112 of the thermal layer. In other embodiments, the thermal layer 106 may have a thickness that is greater than the height of the LEDs 110 such that the LEDs 110 do not protrude outwardly through the surface 112 of the thermal layer 106. The device 100 may include any number of LEDs 110.

LEDs 110 in the illustrated embodiment may output ultraviolet light or any other light incidental to outputting ultraviolet light. Other embodiments may output light at other wavelengths that has sanitizing or other therapeutic properties.

A top layer 114 may be arranged on the thermal layer 112. Top layer 114 may be formed from any suitable sheet material such as a plastic woven fabric or any other type of suitable synthetic or natural material. Top sheet 114 may comprise a number of orifices that are covered with screen material 116. The orifices generally correspond to the locations of the light emitting diodes 110. Screen material 116 may partially obscure the diodes so as to protect a user from touching warm LEDs 110 while allowing light to pass through the screens 116.

Each of the layers 102, 104, 106, and 114 may be connected together using any suitable method or methods, including for example stitching using adhesives or a welding process.

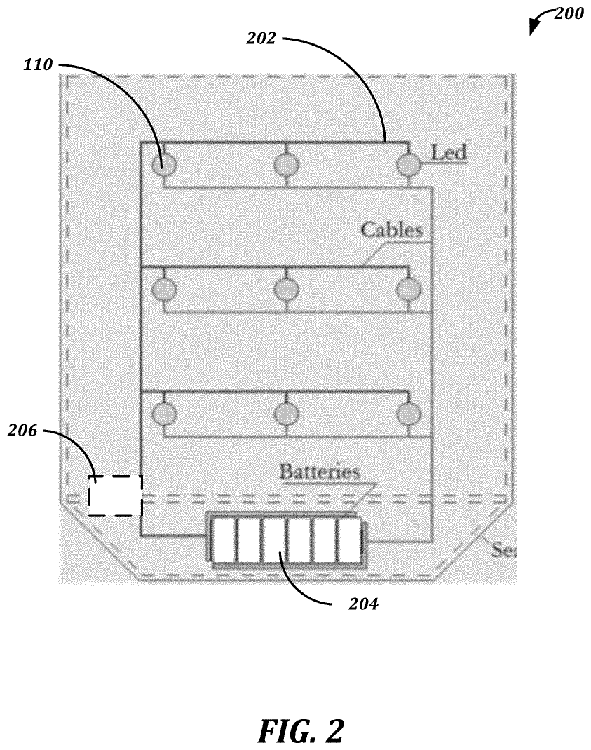

FIG. 2 illustrates a circuit 200 that may include light emitting diodes 110, wires 202, and batteries 204 to form the circuit 200. In the illustrated example embodiment, circuit 200 may be an “ideal circuit.” Alternative embodiments may comprise controllers and associated circuitry 206 and switches.

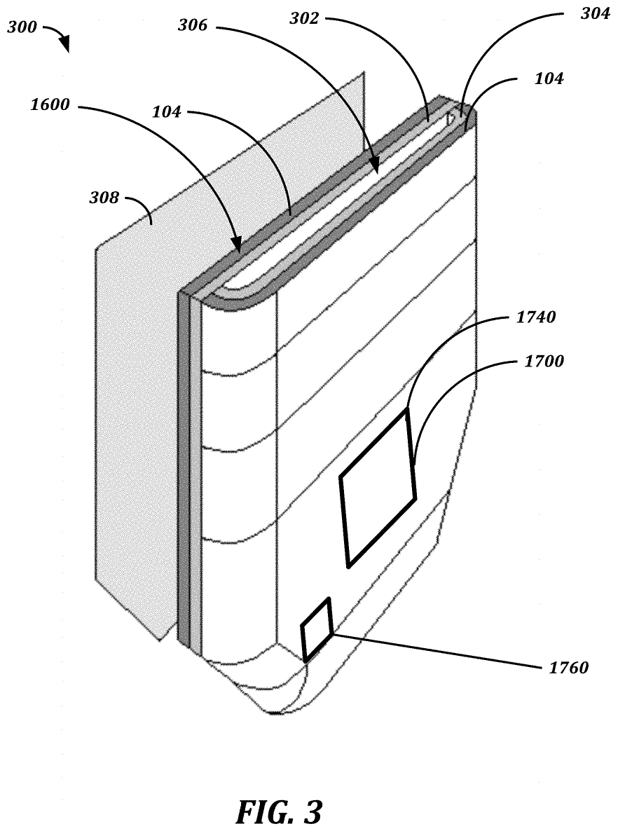

FIG. 3 illustrates a perspective view of an example embodiment of a portion of a pocket shaped disinfectant device 300. The illustrated embodiment may use a first array 302 that is similar to the array 110 of device 100 shown in FIG. 1, and array 304 is also similar to array 110. The array 302 and 304 may be arranged to define a pocket that has an inner cavity 306. A backing layer 308 may be attached to the device 300 to attach the device 300 to a garment or other suitable object.

Interior pocket 306 may be lined with the top sheet 114 (of FIG. 1). LEDs 110 may shine through screen material 112 (of FIG. 1) to illuminate the interior of the pocket and to direct ultraviolet light on opposing sides of the hand of the user.

The pocket shaped disinfectant device 300 and/or the device 100 may be configured to actuate for a predetermined amount of time.

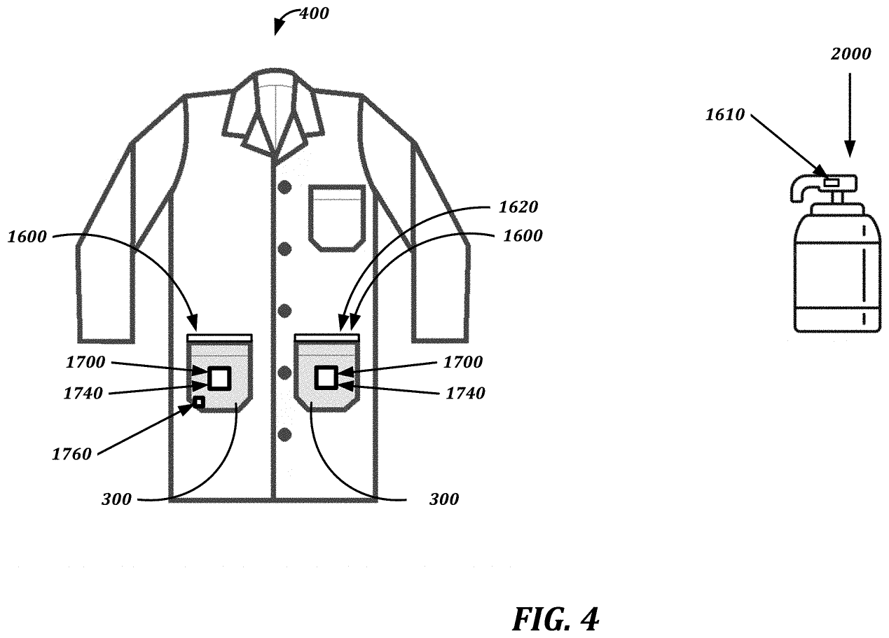

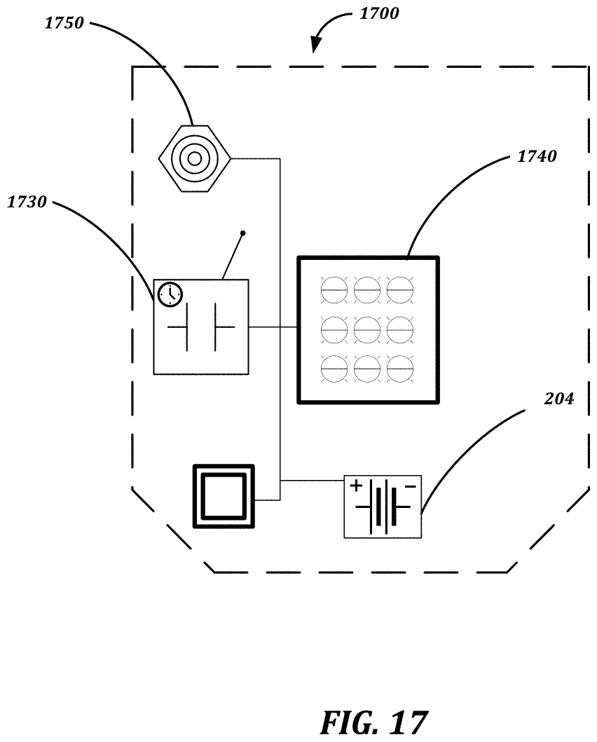

As shown in FIGS. 3 and 4, the device 100 and/or the pocket shaped disinfectant device 300 may, in some embodiments, comprise a sensor 1600. The sensor 1600 may be used to detect one or more objects (e.g., a hand of the user) entering the inner cavity 306. In some embodiments, the sensor 1600 may be configured to actuate and/or be operative to transmit a signal and/or electronic communication responsive to the sensor 1600 detecting one or more objects (e.g., a hand of the user) entering the inner cavity 306. In further embodiments, the sensor 1600 may be coupled to a light switch 1730 (shown at least in FIG. 17). By way of nonlimiting example, the sensor 1600 may be embodied as, but not limited to, for example:
a. a reed switch,
b. a motion sensor,
c. a proximity (and/or geolocational) sensor,
d. a bacteria detection sensor, and/or
e. any other suitable sensor.

In further embodiments, the sensor 1600 may be embodied as a plurality of sensors such as, but not limited to, a first sensor 1610 and a second sensor 1620 (as shown at least in FIG. 4). In some embodiments, the first sensor 1610 and/or the second sensor 1620 may be configured to actuate and/or transmit a signal responsive to the first sensor 1610 and the second sensor 1620 being within a predetermined threshold distance of one another. In some embodiments the threshold distance may be such that a user wearing the second sensor 1620 must physically contact an external disinfecting device 2000 (shown in FIG. 4) housing the first sensor 1610 for the signal to be transmitted. In some embodiments, actuation of the external disinfecting device 2000 may be required to allow the first sensor 1610 to communicate with the second sensor 1620.

By way of nonlimiting example, in one embodiment, the first sensor 1610 may be embodied as a first proximity sensor secured to a portion of an external disinfecting device (configured to administer sanitizing fluid and/or other sanitizing applications) 2000, and the second sensor 1620 may be embodied as a second proximity sensor secured to a garment (and/or the pocket shaped disinfectant device 300 and/or the device 100) of a user. A signal may be transmitted from one or more of the first sensor 1610 and the second sensor 1620 to a lighting apparatus 1700 responsive to the first sensor 1610 and the second sensor 1620 being within a predetermined geolocational distance from one another. In this example, the signal transmission may be an indication that the external disinfecting device 2000 is being used by the user and/or may further indicate a predetermined timeframe of cleanliness of the user after use of the external disinfecting device 2000.

As shown in FIGS. 3 and 4, the device 100 and/or the pocket shaped disinfectant device 300 may, in some embodiments, comprise a lighting apparatus 1700 (further illustrated in FIG. 17). The lighting apparatus 1700 may be used to provide an indication as to cleanliness of the user and/or hands of the user. In some embodiments, the lighting apparatus 1700 may be disposed on an outer surface of the pocket shaped disinfectant device 300. In further embodiments, the lighting apparatus 1700 may be in operative communication with the pocket shaped disinfectant device 300.

In some embodiments, the lighting apparatus 1700 may comprise a display means (and/or a visual indicator, and/or a visual display) 1740. The display means 1740 may be used to visually shine and/or display indications of cleanliness and/or disinfection. As a particular example, the display means 1740 may be configured to display at least one of a clean visual indicator (e.g., indicating that hands of a user are clean or have recently been sanitized) or an unclean visual indicator (e.g., indicating that hands of a user are unclean or that a predetermined amount of time has elapsed since the user last sanitized hands).

In further embodiments, the lighting apparatus 1700 may comprise a clean visual indicator. The clean visual indicator may be used to visually display an indication that the user and/or the hands of the user are clean (via the display means 1740). The clean visual indicator may be embodied as, for example, but not limited to, a visual light color such as green.

In further embodiments, the lighting apparatus 1700 may comprise an unclean visual indicator. The unclean visual indicator may be used to visually display an indication that the user and/or the hands of the user are unclean (via the display means 1740). The unclean visual indicator may be embodied as, for example, but not limited to, a visual light color such as red.

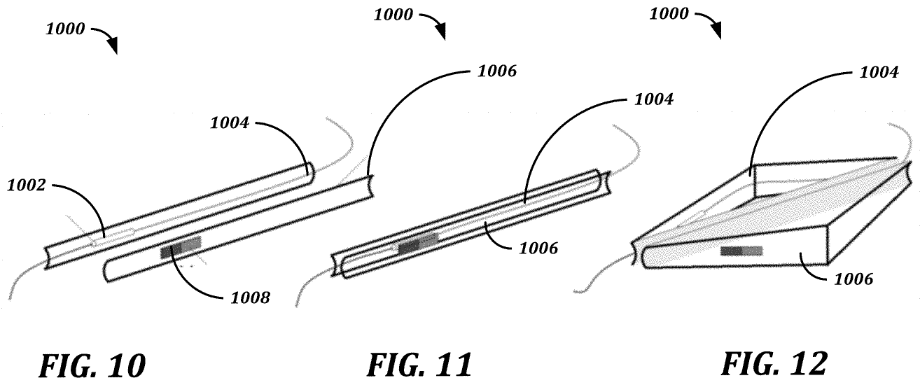

In further embodiments, the lighting apparatus 1700 may comprise the light switch (and/or timing switch) 1730, shown at least in FIG. 17. The light switch 1730 may be used to power, activate, actuate, change color, and/or change the visual representation of the lighting apparatus 1700. In some embodiments, the light switch 1730 may act as similarly to, and/or be interchangeable with, switch 1000 and any components thereof (illustrated at least in FIGS. 10-12). In further embodiments, the light switch 1730 may, responsive to a predetermined amount of time passing and a disinfecting portion not being actuated (e.g., the sensor 1600 not being actuated), display the unclean visual indicator and stop display of the clean visual indicator (and/or convert the clean visual indicator to the unclean visual indicator). In further embodiments, the light switch 1730 may, responsive to the disinfecting portion being activated (e.g., actuation of the sensor 1600), display the clean visual indicator and stop display of the unclean visual indicator (convert the unclean visual indicator to the clean visual indicator).

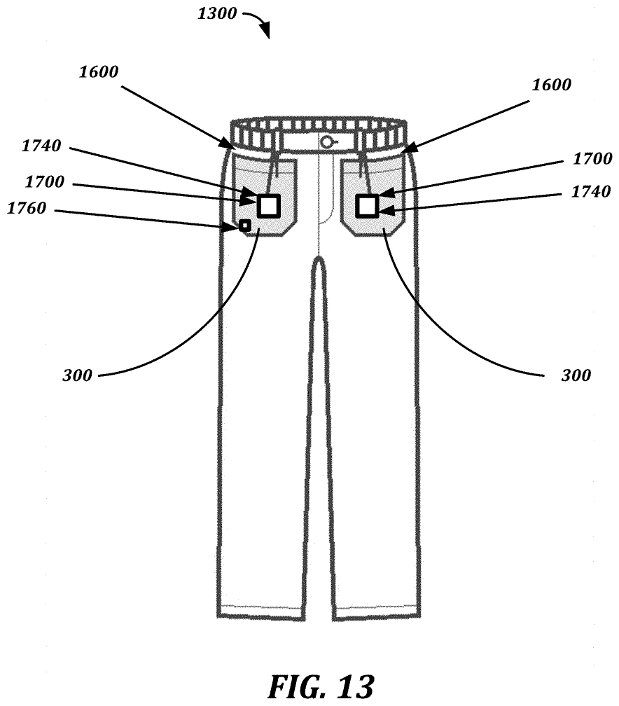

In further embodiments, the lighting apparatus 1700 (shown at least in in FIGS. 3 and 4) may comprise a manual control system 1760 (shown at least in FIG. 13). The manual control system 1760 may be used to override, adjust, and/or control at least a portion of any of the components of the lighting apparatus 1700.

In further embodiments, the pocket shaped disinfectant device 300 and/or the device 100 may comprise a vibrational element 1750, illustrated at least in FIG. 17. The vibrational element 1750 may be used to vibrate at least a portion of the pocket shaped disinfectant device 300 and/or the device 100. In some embodiments, the vibrational element 1750 is configured to vibrate during actuation of the pocket shaped disinfectant device 300 and/or the device 100. Additionally, or alternatively, the vibrational element 1750 may be configured to vibrate in response to the predetermined amount of time passing and the disinfecting portion not being actuated (e.g., as a reminder to the user to disinfect).

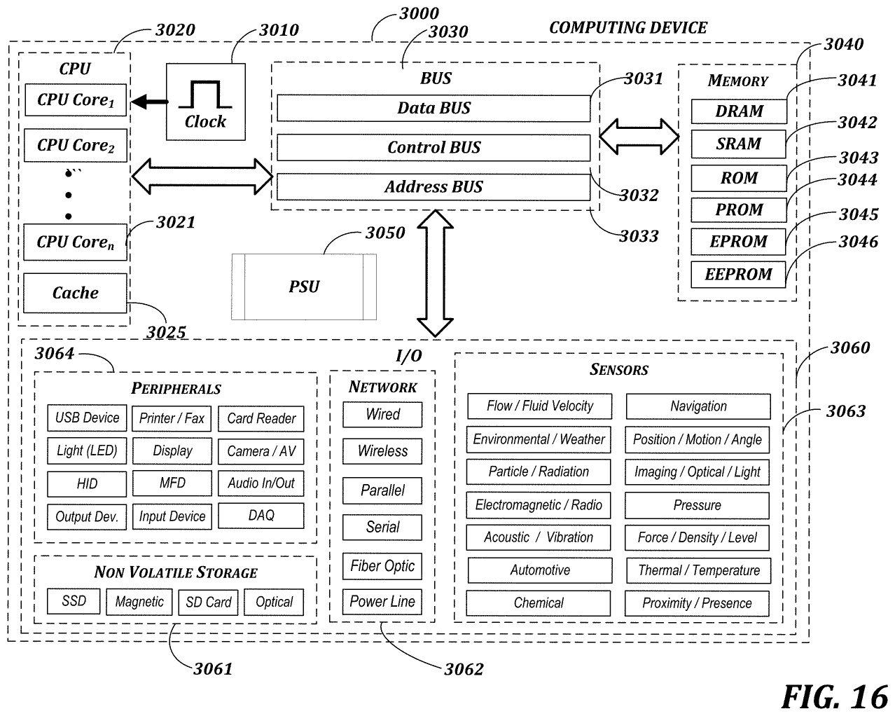

It is noted that any of the aforementioned elements and/or components may be operated with and/or in communication with a computing device 3000 (shown at least in FIG. 16).

FIG. 4 illustrates a garment 400 that includes the pocket shaped device 300. The pocket shaped device 300 may be attached to the garment 400 using any desired attachment component, for example, hook and loop fasteners, stitching, adhesives, snaps, buttons, or other suitable fasteners.

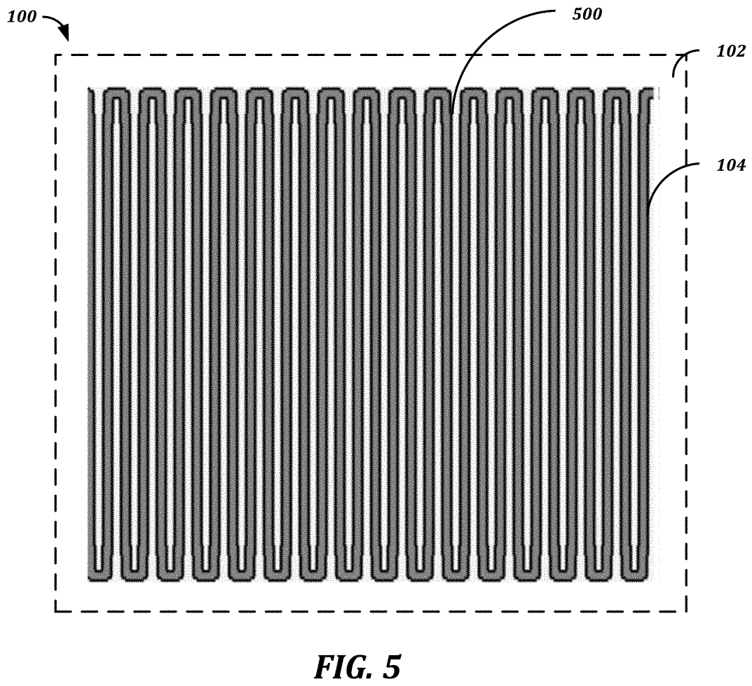

FIGS. 5-8 illustrate a method of manufacturing the device 100. FIG. 5 illustrates thermal reservoir 104 attached to backing sheet 102. Thermal reservoir 104 in the illustrated embodiment may comprise a number of flexible tubes 500 that may be filled with a fluid, such as, for example, water, oil, or other fluid. In various aspects, flexible tubes 500 may comprise cooling channels which may be operative to dissipate heat from LEDs 110.

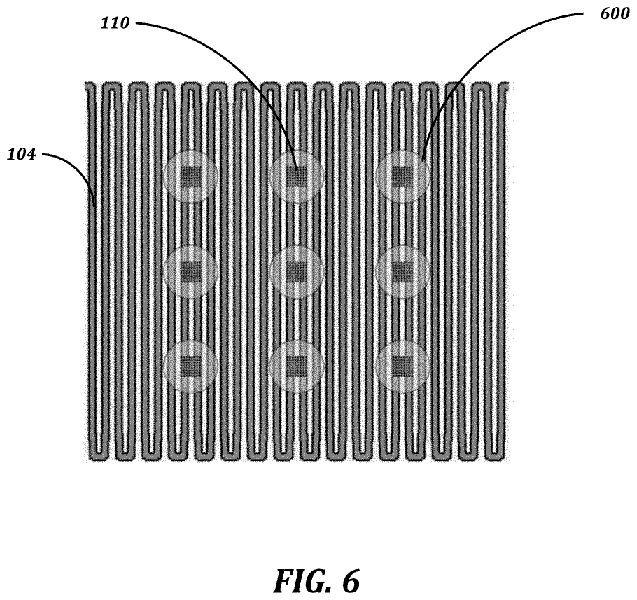

FIG. 6 illustrates a top view of the deposition of LEDs 110 and heatsinks 600 on the reservoir layer 104. LEDs 110 are connected to the thermal reservoir 104 by heatsinks 600. Heatsinks 600 are operative to conduct heat from the LEDs 110 to the thermal reservoir. Heatsinks 600 may be formed from any suitable material that conducts heat and is substantially flexible, such as, for example, a metallic material such as copper, aluminum, and silver. Heatsinks 600 may be arranged in any suitable shape such as a circular or spiral shape.

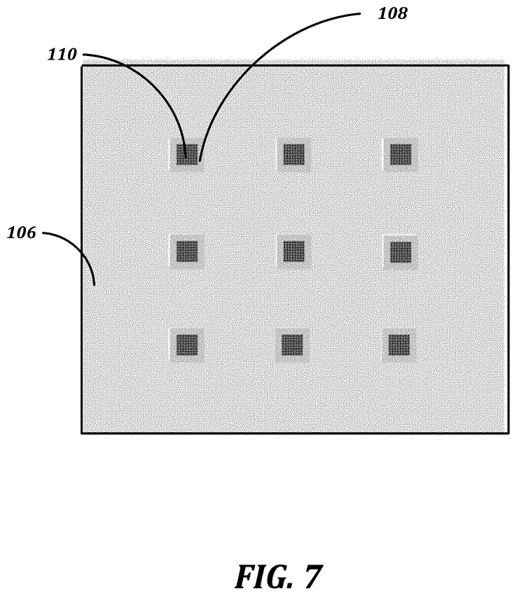

FIG. 7 illustrates a top view of the resulting structure following the arrangement of thermal (insulating) layer 106 on heat reservoir 104. Portions of LEDs 110 are exposed via orifices 108 in thermal layer 106.

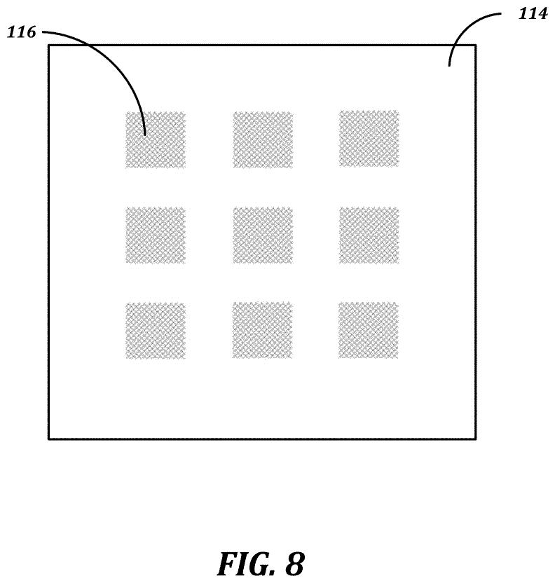

FIG. 8 illustrates a top view of the resulting structure following the application of the top layer 114. Top layer 114 includes screens 116 which protect the user from touching warm LEDs 110 while allowing light to pass through the screens 116 to be emitted into the pocket onto the target surface, such as a user's hand.

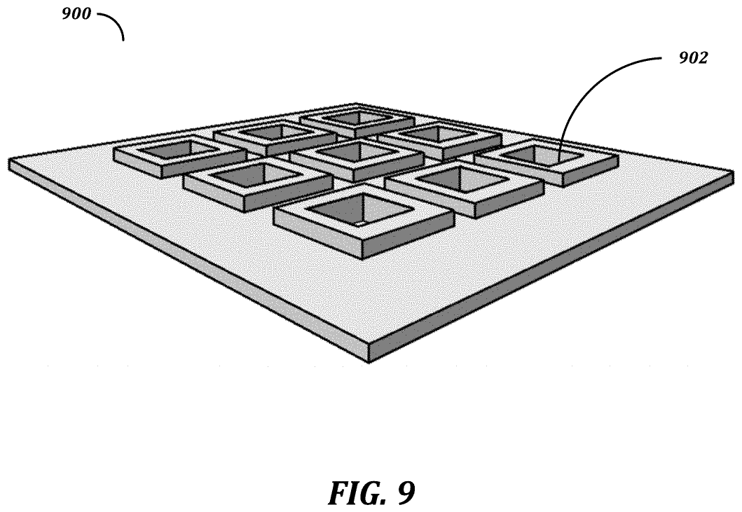

FIG. 9 illustrates a plan view of an alternate embodiment of a thermal layer 900. Thermal layer 900 includes orifices one of the having raised rims 902.

FIGS. 10-12 illustrate a perspective view of the operation of an example of a switch device 1000 that may be used to operate the pocket device 300 shown in FIG. 3 when placed on opposing sides of the pocket. Switch device 1000 may be arranged proximate to the opening of the pocket such that the switch state is changed when an object such as, for example, a hand enters the pocket.

FIG. 10 illustrates a reed switch 1002 that is attached to a flexible and elastic component like a metal portion 1004. A magnet 1008 is attached to a second flexible and elastic component like a metal portion 1006.

FIG. 11 illustrates the arrangement of flexible metal portions 1004 and 1006 when set in a substantially parallel position. Magnet 1008 is proximate to the reed switch 1002.

FIG. 12 illustrates the flexing deformation of the flexible components, metal pieces 1004 and 1006. The deformation allows the hand of a user to enter into the pocket between the bent flexible components 1004 and 1006 while also separating the reed switch 1002 from magnets 1008. In the illustrated example embodiment, when the reed switch 1002 is separated from magnets 1008, such as when the hand of a user deforms the flexible components 1006, LEDs 110 (of FIG. 1) illuminate the interior of the pocket, such as inner cavity of pocket 306, and the contents of the pocket including the hands of the user.

FIG. 13 illustrates an embodiment of a trouser 1300 garment. The trousers 1300 includes features for receiving and retaining the pocket shaped device 300 and/or device 100 such as, for example, hook and loop fasteners, snaps, buttons, or any other suitable attachment arrangement.

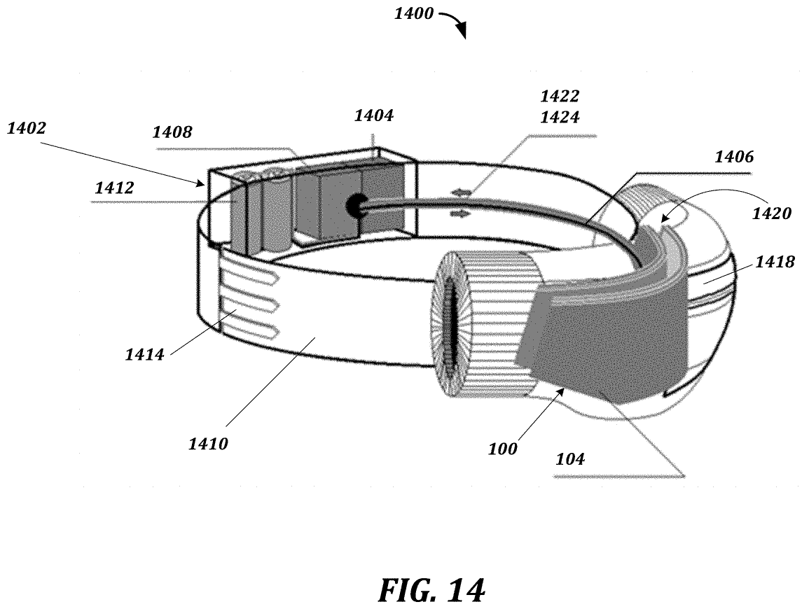

FIG. 14 illustrates an embodiment of a device 1400.

In some embodiments, device 1400 may comprise a liquid exchange portion 1402. In some embodiments, liquid exchange portion 1402 may be configured to connect to a strap portion 1410.

In some embodiments, liquid exchange portion 1402 may comprise a first thermal reservoir 1404. In some embodiments, thermal reservoir 1404 may comprise a number of flexible tubes and/or reservoir that may be filled with a fluid, such as, for example, water, oil, or other fluid.

In further embodiments, liquid exchange portion 1402 may comprise a liquid circulation channel 1406. In some embodiments, liquid circulation channel 1406 may be configured to connect first thermal reservoir 1404 to a second thermal reservoir 104. In further embodiments, liquid circulation channel 1406 may comprise at least one ingress channel 1422 and at least one egress channel 1424. In some aspects, liquid circulation channel 1406 may be contained within strap portion 1410. In other aspects, liquid circulation channel 1406 may be disposed on a surface of strap portion 1410.

In further embodiments, liquid exchange portion 1402 may comprise a liquid pumping device 1408. In some embodiments, liquid pumping device 1408 may be configured to circulate liquid, via liquid circulation channel 1406, between first thermal reservoir 1404 and second thermal reservoir 104.

In further embodiments, liquid exchange portion 1402 may comprise a power source 1412, such as power supply or batteries or the like. In some embodiments, power source 1412 may provide power to device 1400 and components, such as device 100, and/or liquid pumping device 1408.

In further embodiments, device 1400 may comprise strap portion 1410. Strap portion 1410 may be used to secure device 1400 on a user. In some embodiments, strap portion 1410 may comprise a securing means 1414. In further aspects, the securing means may comprise any desired securing mechanism, such as, for example, a cord or strap lock, zipper, buttons, snaps, clips, hooks, fasteners, and combinations thereof.

In further embodiments, device 1400 may comprise a casing or internal housing 1418 configured to contain or house various components of the device. In some embodiments, internal casing 1418 may connect to strap portion 1410. In other embodiments, casing 1418 may be tubular in shape with two opposed opening to allow entry into the interior, such as inserting a hand therein.

In further embodiments, device 1400 may comprise a disinfecting portion 1420. In some embodiments, disinfecting portion 1420 may comprise at least one disinfecting device disclosed herein, for example, device 100 or 300 or a device having a substantially similar configuration. In other embodiments, disinfecting portion 1420 may include a plurality of said devices. In further embodiments, disinfecting portion 1420 may be connected to or otherwise contained within casing 1418. In further embodiments, disinfecting portion 1420 may be arranged to form a pocket. To this end, the interior of said formed pocket is configured to be illuminated by the devices to direct ultraviolet light therein, such as onto opposing sides of the hand of the user when placed inside.

In some embodiments, device 1400 may comprise and/or integrate at least a portion of the components of sensor 1600 and/or the lighting apparatus 1700 and/or perform similar functional operation thereof.

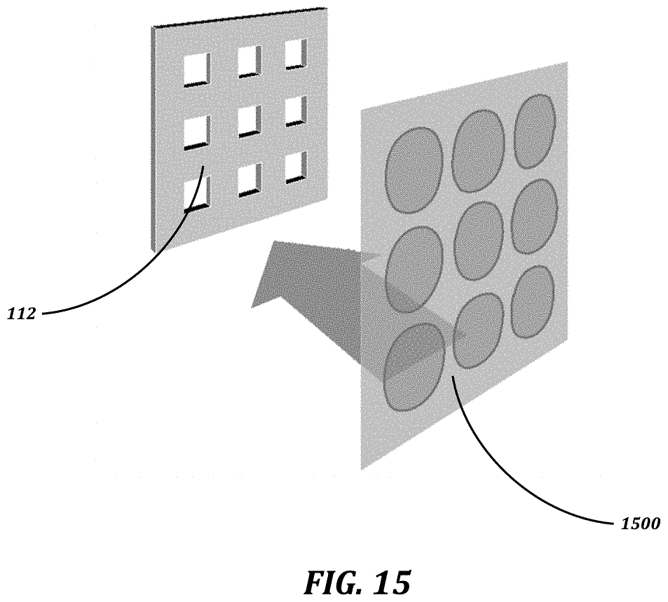

FIG. 15 illustrates an alternate top layer 1500. In some embodiments, alternate top layer 1500 may be arranged on thermal layer 112. In further embodiments, alternate top layer 1500 may be made from a flexible material. In further embodiments, alternate top layer 1500 may be made from a transparent and/or translucent material. In further embodiments, alternate top layer 1500 may be made from a material capable of passing UV-C light through top layer 1500.

The embodiments described above include a method and device for disinfecting the hands of a user that may be worn as clothing. Such a device can provide a pocket to receive and illuminate the hands of a user with a disinfecting light.

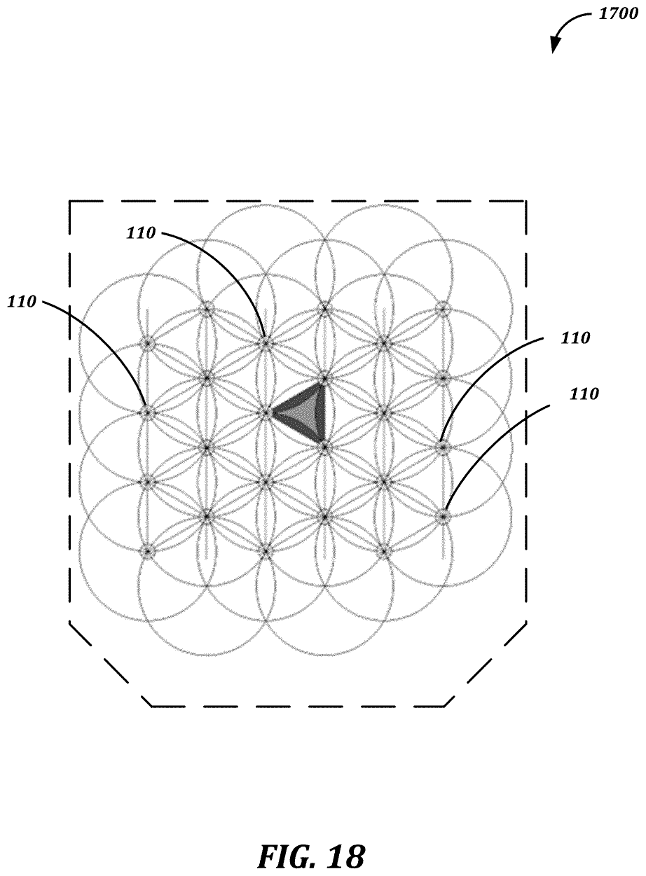

FIG. 18 illustrates an arrangement and/or distribution of a plurality of the LEDs 110. In some embodiments, each LED 110 may have an irradiation field with optimal or approximately optimal disinfecting effect of objects in a range of 2 mm-18 mm from a surface of the LED 110. The arrangement of the plurality of LEDs 110 may allow for a superposition of multiple (e.g., 3 to 4) irradiation fields at any given location within the proximity of the plurality of LEDs 110. In some embodiments, the plurality of LEDs 110 may be positioned relative to one another at a triangular distribution. In further embodiments, the plurality of LEDs 110 may be positioned relative to one another at a square distribution, for example. In any distribution case, the polar angle is used for the irradiation to define the area of influence of each LED. In some embodiments, the plurality of LEDs 110 may be positioned relative to one another at a polar angle of 55 degrees. While FIG. 18 shows one potential arrangement of the plurality of LEDs 110, it should be noted that more or fewer LEDs and/or differing arrangements of the LEDs may be used without departing from the scope of the invention. The arrangement and/or distribution of a plurality of the LEDs 110 shown in FIG. 18 may be used for any of the aforementioned garments and/or pockets such as, but not limited to, the pocket shaped disinfectant device 300, the device 100, the garment 400, the garment 1300, and the device 1400.

At least a portion of the device 100 and/or the pocket shaped disinfectant device 300 may be embodied as and/or operated by, for example, but not be limited to, a website, a web application, a desktop application, backend application, and a mobile application compatible with a computing device 3000. The computing device 3000 may comprise, but not be limited to the following:

Mobile computing device, such as, but is not limited to, a laptop, a tablet, a smartphone, a drone, a wearable, an embedded device, a handheld device, an Arduino, an industrial device, or a remotely operable recording device;

A supercomputer, an exa-scale supercomputer, a mainframe, or a quantum computer;

A minicomputer, wherein the minicomputer computing device comprises, but is not limited to, an IBM AS400/iSeries/System I, A DEC VAX/PDP, a HP3000, a Honeywell-Bull DPS, a Texas Instruments TI-990, or a Wang Laboratories VS Series;

A microcomputer, wherein the microcomputer computing device comprises, but is not limited to, a server, wherein a server may be rack mounted, a workstation, an industrial device, a raspberry pi, a desktop, or an embedded device;

At least a portion of the device 100 may be hosted on a centralized server or a cloud computing service. Although the aforementioned methods have been described to be performed by a computing device 3000, it should be understood that, in some embodiments, different operations may be performed by a plurality of the computing devices 3000 in operative communication with at least one network.

Embodiments of the present disclosure may comprise a system having a central processing unit (CPU) 3020, a bus 3030, a memory unit 3040, a power supply unit (PSU) 3050, and one or more Input/Output (I/O) units. The CPU 3020 coupled to the memory unit 3040 and the plurality of I/O units 3060 via the bus 3030, all of which are powered by the PSU 3050. It should be understood that, in some embodiments, each disclosed unit may actually be a plurality of such units for the purposes of redundancy, high availability, and/or performance. The combination of the presently disclosed units is configured to perform the stages any method disclosed herein.

FIG. 16 is a block diagram of a system including computing device 3000. Consistent with an embodiment of the disclosure, the aforementioned CPU 3020, the bus 3030, the memory unit 3040, a PSU 3050, and the plurality of I/O units 3060 may be implemented in a computing device, such as computing device 3000 of FIG. 16. Any suitable combination of hardware, software, or firmware may be used to implement the aforementioned units. For example, the CPU 3020, the bus 3030, and the memory unit 3040 may be implemented with computing device 3000 or any of other computing devices 3000, in combination with computing device 3000. The aforementioned system, device, and components are examples and other systems, devices, and components may comprise the aforementioned CPU 3020, the bus 3030, the memory unit 3040, consistent with embodiments of the disclosure.

At least one computing device 3000 may be embodied as any of the computing elements illustrated in all of the attached figures and/or be in operative communication with (or embedded in) any of the aforementioned components of the device 100, the circuit 200, the pocket shaped disinfectant device 300, the garment 400, the flexible tubes 500, the heatsinks 600, the thermal layer 900, the switch device 1000, the trousers 1300, the device 1400, the top layer 1500, the sensor 1600, and/or the lighting apparatus 1700. A computing device 3000 does not need to be electronic, nor even have a CPU 3020, nor bus 3030, nor memory unit 3040. The definition of the computing device 3000 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 3000, especially if the processing is purposeful.

With reference to FIG. 16, a system consistent with an embodiment of the disclosure may include a computing device, such as computing device 3000. In a basic configuration, computing device 3000 may include at least one clock module 3010, at least one CPU 3020, at least one bus 3030, and at least one memory unit 3040, at least one PSU 3050, and at least one I/O 3060 module, wherein I/O module may be comprised of, but not limited to a non-volatile storage sub-module 3061, a communication sub-module 3062, a sensors sub-module 3063, and a peripherals sub-module 3064.

A system consistent with an embodiment of the disclosure the computing device 3000 may include the clock module 3010 may be known to a person having ordinary skill in the art as a clock generator, which produces clock signals. Clock signal is a particular type of signal that oscillates between a high and a low state and is used like a metronome to coordinate actions of digital circuits. Most integrated circuits (Ics) of sufficient complexity use a clock signal in order to synchronize different parts of the circuit, cycling at a rate slower than the worst-case internal propagation delays. The preeminent example of the aforementioned integrated circuit is the CPU 3020, the central component of modern computers, which relies on a clock. The only exceptions are asynchronous circuits such as asynchronous CPUs. The clock 3010 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 4 wires.

Many computing devices 3000 use a “clock multiplier” which multiplies a lower frequency external clock to the appropriate clock rate of the CPU 3020. This allows the CPU 3020 to operate at a much higher frequency than the rest of the computer, which affords performance gains in situations where the CPU 3020 does not need to wait on an external factor (like memory 3040 or input/output 3060). Some embodiments of the clock 3010 may include dynamic frequency change, where, the time between clock edges can vary widely from one edge to the next and back again.

A system consistent with an embodiment of the disclosure of the computing device 3000 may include the CPU unit 3020 comprising at least one CPU Core 3021. A plurality of CPU cores 3021 may comprise identical CPU cores 3021, such as, but not limited to, homogeneous multi-core systems. It is also possible for the plurality of CPU cores 3021 to comprise different CPU cores 3021, such as, but not limited to, heterogeneous multi-core systems, big.LITTLE systems and some AMD accelerated processing units (APU). The CPU unit 3020 reads and executes program instructions which may be used across many application domains, for example, but not limited to, general purpose computing, embedded computing, network computing, digital signal processing (DSP), and graphics processing (GPU). The CPU unit 3020 may run multiple instructions on separate CPU cores 3021 at the same time. The CPU unit 3020 may be integrated into at least one of a single integrated circuit die and multiple dies in a single chip package. The single integrated circuit die and multiple dies in a single chip package may contain a plurality of other aspects of the computing device 3000, for example, but not limited to, the clock 3010, the CPU 3020, the bus 3030, the memory 3040, and I/O 3060.

The CPU unit 3020 may contain cache 3022 such as, but not limited to, a level 1 cache, level 2 cache, level 3 cache or combination thereof. The aforementioned cache 3022 may or may not be shared amongst a plurality of CPU cores 3021. The cache 3022 sharing comprises at least one of message passing and inter-core communication methods may be used for the at least one CPU Core 3021 to communicate with the cache 3022. The inter-core communication methods may comprise, but not limited to, bus, ring, two-dimensional mesh, and crossbar. The aforementioned CPU unit 3020 may employ symmetric multiprocessing (SMP) design.

The plurality of the aforementioned CPU cores 3021 may comprise soft microprocessor cores on a single field programmable gate array (FPGA), such as semiconductor intellectual property cores (IP Core). The plurality of CPU cores 3021 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 3021, for example, but not limited to Instruction-level parallelism (ILP) such as, but not limited to, superscalar pipelining, and Thread-level parallelism (TLP).

Consistent with the embodiments of the present disclosure, the aforementioned computing device 3000 may employ a communication system that transfers data between components inside the aforementioned computing device 3000, and/or the plurality of computing devices 3000. The aforementioned communication system will be known to a person having ordinary skill in the art as a bus 3030. The bus 3030 may embody internal and/or external plurality of hardware and software components, for example, but not limited to a wire, optical fiber, communication protocols, and any physical arrangement that provides the same logical function as a parallel electrical bus. The bus 3030 may comprise at least one of, but not limited to a parallel bus, wherein the parallel bus carry data words in parallel on multiple wires, and a serial bus, wherein the serial bus carry data in bit-serial form. The bus 3030 may embody a plurality of topologies, for example, but not limited to, a multidrop/electrical parallel topology, a daisy chain topology, and a connected by switched hubs, such as USB bus. The bus 3030 may comprise a plurality of embodiments, for example, but not limited to:
Internal data bus (data bus) 3031/Memory bus
Control bus 3032
Address bus 3033
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OcuLink [Optical Copper{Cu} Link]), Express Card, AdvancedTCA, AMC, Universal 10, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and eXtensible Host Controller Interface (xHCI).

Consistent with the embodiments of the present disclosure, the aforementioned computing device 3000 may employ hardware integrated circuits that store information for immediate use in the computing device 3000, know to the person having ordinary skill in the art as primary storage or memory 3040. The memory 3040 operates at high speed, distinguishing it from the non-volatile storage sub-module 3061, which may be referred to as secondary or tertiary storage, which provides slow-to-access information but offers higher capacities at lower cost. The contents contained in memory 3040, may be transferred to secondary storage via techniques such as, but not limited to, virtual memory and swap. The memory 3040 may be associated with addressable semiconductor memory, such as integrated circuits consisting of silicon-based transistors, used for example as primary storage but also other purposes in the computing device 3000. The memory 3040 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 3041, Static Random-Access Memory (SRAM) 3042, CPU Cache memory 3025, Advanced Random-Access Memory (A-RAM), and other types of primary storage such as Random-Access Memory (RAM).
Non-volatile memory which can retain stored information even after power is removed, for example, but not limited to, Read-Only Memory (ROM) 3043, Programmable ROM (PROM) 3044, Erasable PROM (EPROM) 3045, Electrically Erasable PROM (EEPROM) 3046 (e.g., flash memory and Electrically Alterable PROM [EAPROM]), Mask ROM (MROM), One Time Programmable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D Xpoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).

Consistent with the embodiments of the present disclosure, the aforementioned computing device 3000 may employ the communication system between an information processing system, such as the computing device 3000, and the outside world, for example, but not limited to, human, environment, and another computing device 3000. The aforementioned communication system will be known to a person having ordinary skill in the art as I/O 3060. The I/O module 3060 regulates a plurality of inputs and outputs with regard to the computing device 3000, wherein the inputs are a plurality of signals and data received by the computing device 3000, and the outputs are the plurality of signals and data sent from the computing device 3000. The I/O module 3060 interfaces a plurality of hardware, such as, but not limited to, non-volatile storage 3061, communication devices 3062, sensors 3063, and peripherals 3064. The plurality of hardware is used by the at least one of, but not limited to, human, environment, and another computing device 3000 to communicate with the present computing device 3000. The I/O module 3060 may comprise a plurality of forms, for example, but not limited to channel I/O, port mapped I/O, asynchronous I/O, and Direct Memory Access (DMA).

Consistent with the embodiments of the present disclosure, the aforementioned computing device 3000 may employ the non-volatile storage sub-module 3061, which may be referred to by a person having ordinary skill in the art as one of secondary storage, external memory, tertiary storage, off-line storage, and auxiliary storage. The non-volatile storage sub-module 3061 may not be accessed directly by the CPU 3020 without using intermediate area in the memory 3040. The non-volatile storage sub-module 3061 does not lose data when power is removed and may be two orders of magnitude less costly than storage used in memory module, at the expense of speed and latency. The non-volatile storage sub-module 3061 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (3061) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 3000 may employ the communication sub-module 3062 as a subset of the I/O 3060, which may be referred to by a person having ordinary skill in the art as at least one of, but not limited to, computer network, data network, and network. The network allows computing devices 3000 to exchange data using connections, which may be known to a person having ordinary skill in the art as data links, between network nodes. The nodes comprise network computer devices 3000 that originate, route, and terminate data. The nodes are identified by network addresses and can include a plurality of hosts consistent with the embodiments of a computing device 3000. The aforementioned embodiments include, but not limited to personal computers, phones, servers, drones, and networking devices such as, but not limited to, hubs, switches, routers, modems, and firewalls.

Two nodes can be said are networked together, when one computing device 3000 is able to exchange information with the other computing device 3000, whether or not they have a direct connection with each other. The communication sub-module 3062 supports a plurality of applications and services, such as, but not limited to World Wide Web (WWW), digital video and audio, shared use of application and storage computing devices 3000, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 4 [Ipv4], and Internet Protocol version 6 [Ipv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 3062 may comprise a plurality of size, topology, traffic control mechanism and organizational intent. The communication sub-module 3062 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G, 4G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 3000 may employ the sensors sub-module 3063 as a subset of the I/O 3060. The sensors sub-module 3063 comprises at least one of the devices, modules, and subsystems whose purpose is to detect events or changes in its environment and send the information to the computing device 3000. Sensors are sensitive to the measured property, are not sensitive to any property not measured, but may be encountered in its application, and do not significantly influence the measured property. The sensors sub-module 3063 may comprise a plurality of digital devices and analog devices, wherein if an analog device is used, an Analog to Digital (A-to-D) converter must be employed to interface the said device with the computing device 3000. The sensors may be subject to a plurality of deviations that limit sensor accuracy. The sensors sub-module 3063 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 3000 may employ the peripherals sub-module 3064 as a subset of the I/O 3060. The peripheral sub-module 3064 comprises ancillary devices uses to put information into and get information out of the computing device 3000. There are 3 categories of devices comprising the peripheral sub-module 3064, which exist based on their relationship with the computing device 3000, input devices, output devices, and input/output devices. Input devices send at least one of data and instructions to the computing device 3000. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two-dimensional mice vs three-dimensional mice used for Computer-Aided Design (CAD) applications.

Output devices provide output from the computing device 3000. Output devices convert electronically generated information into a form that can be presented to humans. Input/output devices perform both input and output functions. It should be understood by a person having ordinary skill in the art that the ensuing are non-limiting embodiments of the aforementioned peripheral sub-module 3064:
Input Devices
   Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
   High degree of freedom devices, that require up to six degrees of freedom such as, but not limited to, camera gimbals, Cave Automatic Virtual Environment (CAVE), and virtual reality systems.
   Video Input devices are used to digitize images or video from the outside world into the computing device 3000. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
   Audio input devices are used to capture sound. In some cases, an audio output device can be used as an input device, in order to capture produced sound. Audio input devices allow a user to send audio signals to the computing device 3000 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
   Data AcQuisition (DAQ) devices convert at least one of analog signals and physical parameters to digital values for processing by the computing device 3000. Examples of DAQ devices may include, but not limited to, Analog to Digital Converter (ADC), data logger, signal conditioning circuitry, multiplexer, and Time to Digital Converter (TDC).
Output Devices may further comprise, but not be limited to:
   Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).
   Printers, such as, but not limited to, inkjet printers, laser printers, 3D printers, solid ink printers and plotters.
   Audio and Video (AV) devices, such as, but not limited to, speakers, headphones, amplifiers and lights, which include lamps, strobes, DJ lighting, stage lighting, architectural lighting, special effect lighting, and lasers.
   Other devices such as Digital to Analog Converter (DAC)
Input/Output Devices may further comprise, but not be limited to, touchscreens, networking device (e.g., devices disclosed in network 3062 sub-module), data storage device (non-volatile storage 3061), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present disclosure includes at least the following aspects:

Aspect 1: A device comprising: a thermal reservoir; a light emitting diode (LED); a heat sink connecting the LED to the thermal reservoir; and an insulating layer arranged on the thermal reservoir, the insulating layer including an orifice arranged over the LED such that light from the LED passes through the orifice in the insulating layer.
Aspect 2: The device of aspect 1, further comprising a backing layer arranged on the thermal reservoir.
Aspect 3: The device of aspect 1 or 2, further comprising a front layer arranged on the insulating layer.
Aspect 4: The device of aspect 2, wherein the backing layer comprises a fabric.
Aspect 5: The device of aspect 2, 3 or 4, wherein the front layer includes a mesh portion arranged over the orifice.
Aspect 6: The device of any preceding aspect, wherein the LED is operative to emit a light that disinfects when shone on a surface.
Aspect 7: The device of any preceding aspect, wherein the thermal reservoir includes a liquid disposed in a channel.
Aspect 8: The device of any preceding aspect, further comprising a fastener layer arranged on a backing layer.
Aspect 9: The device of any preceding aspect, wherein the insulating layer is deformable.
Aspect 10: The device of any preceding aspect, wherein the thermal reservoir is deformable.
Aspect 11: The device of any preceding aspect, wherein the thermal reservoir and the insulating layer are connected by stitching.
Aspect 12: The device of any preceding aspect, wherein the device is actuated by a switch comprising: a first flexible portion; a second flexible portion; and a switch arranged on the first flexible portion, the switch operative to change states when the first metal portion is separated from the second flexible portion.
Aspect 13: The device of any preceding aspect, arranged to form a pocket.
Aspect 14: The device of aspect 13, wherein the LED is arranged on a first portion of the pocket and a second LED is arranged on a second portion of the pocket that opposes the first portion of the pocket.
Aspect 15: A garment comprising: a pocket having: a thermal reservoir; a light emitting diode (LED); a heat sink connecting the LED to the thermal reservoir; and an insulating layer arranged on the thermal reservoir, the insulating layer including an orifice arranged over the LED such that light from the LED passes through the orifice in the insulating layer.
Aspect 16: The device or garment of aspect 15, further comprising a backing layer arranged on the thermal reservoir.
Aspect 17: The device or garment of aspect 15, further comprising a front layer arranged on the insulating layer.
Aspect 18: The device or garment of aspect 15, wherein the LED is arranged on a first portion of the pocket and a second LED is arranged on a second portion of the pocket that opposes the first portion of the pocket.
Aspect 19: The device or garment of any preceding aspect, wherein the thermal reservoir is deformable.
Aspect 20: A device comprising: a thermal reservoir; a backing layer arranged on the thermal reservoir; a light emitting diode (LED); a heat sink connecting the LED to the thermal reservoir; an insulating layer arranged on the thermal reservoir, the insulating layer including an orifice arranged over the LED such that light from the LED passes through the orifice in the insulating layer; and a front layer arranged on the insulating layer the front layer includes a mesh portion arranged over the orifice.
Aspect 21: A device comprising: a liquid exchange portion comprising: a first thermal reservoir, a liquid circulation channel configured to connect the first thermal reservoir to a second thermal reservoir, and a liquid pumping device configured to circulate liquid, via the liquid circulation channel, between the first thermal reservoir and the second thermal reservoir; and a disinfecting portion configured to disinfect a surface, the disinfecting portion comprising: a light emitting diode (LED), a heat sink connecting the LED to the second thermal reservoir, an insulating layer arranged on the second thermal reservoir, the insulating layer including an orifice arranged over the LED such that light from the LED passes through the orifice of the insulating layer, and a front layer arranged on the insulating layer and over the orifice.
Aspect 22: The device of any preceding aspect, further comprising a backing layer arranged on the second thermal reservoir, wherein the backing layer comprises a fabric.
Aspect 23: The device of any preceding aspect, further comprising a strap portion including a securing means, the strap configured to secure the device to an object or user.
Aspect 24: The device of aspect 23, wherein the liquid exchange portion is configured to connect to the strap portion.
Aspect 25: The device of aspect 23 or 24, further comprising a casing configured to connect to the strap portion.
Aspect 26: The device of aspect 25, wherein the casing is configured to contain the disinfecting portion.
Aspect 27: The device of any preceding aspect, wherein the LED is operative to emit a light effective to disinfect when shone on a surface.
Aspect 28: The device of any preceding aspect, wherein the second thermal reservoir comprises a liquid disposed in a channel.
Aspect 29: The device of any preceding aspect, further comprising a fastener layer arranged on a backing layer.
Aspect 30: The device of any preceding aspect, wherein the second thermal reservoir and the insulating layer are connected by stitching.
Aspect 31: The device of any preceding aspect, wherein the device is activated or actuated by a switch comprising: a first flexible portion; a second flexible portion; and a switch arranged on the first metal portion, the switch operative to change states when the first metal portion is separated from the second metal portion.
Aspect 32: The device of any preceding aspect, wherein the disinfecting portion is arranged to form a pocket.
Aspect 33: The device of any preceding aspect, wherein the LED is arranged on a first portion of a pocket and a second LED is arranged on a second portion of the pocket that opposes the first portion of the pocket.
Aspect 34: The device of any preceding aspect, wherein the liquid circulation channel comprises an ingress channel and an egress channel.
Aspect 35: A garment comprising: a liquid exchange portion comprising: a first thermal reservoir, a liquid circulation channel configured to connect the first thermal reservoir to a second thermal reservoir; a liquid pumping device configured to circulate liquid, via the liquid circulation channel, between the first thermal reservoir and the second thermal reservoir; and a pocket configured to disinfect a surface, the pocket comprising: the second thermal reservoir; a light emitting diode (LED), the LED being operative to emit a light effective to disinfect a surface when shone thereon; a heat sink connecting the LED to the second thermal reservoir; an insulating layer arranged on the second thermal reservoir, the insulating layer including an orifice arranged over the LED such that light from the LED passes through the orifice in the insulating layer; and a front layer arranged on the insulating layer arranged over the orifice.
Aspect 36: The garment of any preceding aspect, further comprising a backing layer arranged on the second thermal reservoir.
Aspect 37: The garment of any preceding aspect, wherein the garment comprises a garment body; wherein the pocket is connected to a first portion of the garment body and the liquid exchange portion is connected to a second portion of the garment body.
Aspect 38: The garment of any preceding aspect, wherein the LED is arranged on a first portion of the pocket and a second LED is arranged on a second portion of the pocket that opposes the first portion of the pocket.
Aspect 39: The garment of any preceding aspect, wherein the second thermal reservoir and the insulating layer are deformable.
Aspect 40: The garment of any preceding aspect, wherein the liquid circulation channel comprises an ingress channel and an egress channel.
Aspect 41: A method for disinfecting an object, the method comprising:

arranging a disinfecting device forming a pocket comprising an inner cavity;

inserting the object inside the inner cavity of the disinfecting device, the disinfecting device comprising:
a light emitting diode (LED);
a heat sink connecting the LED to a thermal reservoir; and
an insulating layer arranged on the thermal reservoir, the insulating layer including an orifice arranged over the LED such that light from the LED passes through the orifice in the insulating layer, wherein the front layer includes a mesh portion arranged over the orifice; and

illuminating the interior of the disinfecting device such that LED shines on the surface of the object.

Aspect 42: The method of any preceding aspect, wherein inserting the object inside the inner cavity of the disinfecting device comprises separating a first flexible portion and a second flexible portion located at an insertion point of the disinfecting device.
Aspect 43: The method of any preceding aspect, wherein separating the first flexible portion and the second flexible portion comprises activating a switch located on the first flexible portion.
Aspect 44: The method of any preceding aspect, wherein inserting the object inside the disinfecting device comprises deforming the inner cavity of the disinfecting device.
Aspect 45: The method of any preceding aspect, further comprising dispersing heat in the thermal reservoir via channels comprised in the thermal reservoir containing fluid.
Aspect 46: The method of any preceding aspect, wherein illuminating, via the LED, the surface of the object comprises directing ultraviolet light on opposing sides of the object
Aspect 47: A method for disinfecting an object, the method comprising:
inserting an object inside a disinfecting device;
triggering an activation of the disinfecting device, the activation comprising:
illuminating, via a light emitting diode (LED) passing through at least one orifice of an insulating layer, the interior of the disinfecting device, and
illuminating, via the LED, the surface of the object; and transferring heat generated from the LED, via a heat sink connected to the LED, to a thermal reservoir.
   Aspect 48: The method of any preceding aspect, wherein inserting the object inside the disinfecting device comprises separating a first flexible portion and a second flexible portion located at an insertion point of the disinfecting device.
   Aspect 49: The method of any preceding aspect, wherein separating the first flexible portion and the second flexible portion comprises activating a switch located on the first flexible portion.
   Aspect 50: The method of any preceding aspect, wherein inserting the object inside the disinfecting device comprises deforming an inner cavity of the disinfecting device.
   Aspect 51: The method of any preceding aspect, further comprising dispersing heat in the thermal reservoir via channels comprised in the thermal reservoir containing fluid.
   Aspect 52: The method of any preceding aspect, wherein illuminating, via the LED, the surface of the object comprises directing ultraviolet light on opposing sides of the object.
   Aspect 53: The method of any preceding aspect, wherein triggering an activation of the disinfecting device comprises activating a power source configured to power the disinfecting device.
   Aspect 54: The method of any preceding aspect, further comprising: orienting the LED on a first portion of the disinfecting device; and orienting a second LED on a second portion of the disinfecting device that opposes the first portion of the disinfecting device.
   Aspect 55: A device comprising:

at least one opening, the at least one opening being configured receive an object;

an inner cavity within the at least one opening, the inner cavity comprising:
a light emitting diode (LED),
a heat sink connecting the LED to a thermal reservoir, and
an insulating layer arranged on the thermal reservoir, the insulating layer comprising an orifice arranged over the LED such that light from the LED passes through the orifice of the insulating layer; and

a power source configured to power the LED.

Aspect 56: The device of any preceding aspect, wherein the at least one opening comprises a switch connected to a first flexible portion and a second flexible portion, the first flexible portion being configured to releasably secure to the second flexible portion.
Aspect 57: The device any preceding aspect, wherein the switch is configured to activate the LED upon the first flexible portion being separated from the second flexible portion.
Aspect 58: The device of any preceding aspect, wherein the thermal reservoir is deformable.
Aspect 59: The device of any preceding aspect, wherein the LED is arranged on a first portion of the inner cavity and a second LED is arranged on a second portion of the inner cavity that opposes the first portion of the inner cavity.
Aspect 60: The device of any preceding aspect, further comprising a backing layer arranged on the thermal reservoir.
Aspect 61: The device of any preceding aspect, further comprising a fastener layer arranged on the backing layer.
Aspect 62: A device comprising:

at least one opening, the at least one opening being configured receive an object;

an external housing configured to secure to a garment;

an entry portion configured to be in a closed state and an open state;

a switch disposed on the entry portion; and

an internal housing within the at least one opening, the internal housing comprising:
a light emitting diode (LED),
a heat sink connecting the LED to a thermal reservoir, and
an insulating layer arranged on the thermal reservoir, the insulating layer comprising an orifice arranged over the LED such that light from the LED passes through the orifice of the insulating layer.
   Aspect 63: The device of any preceding aspect, wherein the entry portion is configured receive the object.
   Aspect 64: The device of any preceding aspect, wherein the device is activated by the switch.
   Aspect 65: The device of any preceding aspect, wherein the switch is operative to change states upon the entry portion being in the open state.
   Aspect 66: The device of any preceding aspect, wherein the LED is arranged on a first portion of internal housing and a second LED is arranged on a second portion of the internal housing that opposes the first portion of the internal housing.
   Aspect 67: The device of any preceding aspect, further comprising a backing layer arranged on the thermal reservoir, wherein the backing layer comprises a fabric.
   Aspect 68: The device of any preceding aspect, further comprising a further comprising a fastener layer arranged on the backing layer.
   Aspect 69: The device of any preceding aspect, wherein the LED is operative to emit light effective to disinfect when shone on a surface.
   Aspect 70: A device comprising:

a light emitting diode (LED);

a heat sink connecting the LED to a liquid circulation channel, the heat sink being configured to transfer heat from the LED to the liquid circulation channel; and

an insulating layer arranged on a thermal reservoir, the insulating layer including an orifice arranged over the LED such that light from the LED passes through the orifice in the insulating layer.

Aspect 71: The device of any preceding aspect, wherein the liquid circulation channel is configured to channel heat away from the heat sink.
Aspect 72: The device of any preceding aspect, wherein the thermal reservoir and the insulating layer are connected by stitching.
Aspect 73: The device of any preceding aspect, wherein the liquid circulation channel comprises an ingress channel and an egress channel.
Aspect 74: The device of any preceding aspect, wherein the liquid circulation channel is deformable.

The patentable scope of the present disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

The following is claimed is:

1. A garment comprising:
a disinfecting device comprising:
a cavity,
a sensor operative to actuate upon one or more objects entering the cavity, and
a disinfecting portion configured to disinfect the one or more objects within the cavity, the disinfecting portion being actuated upon receiving an indication from the sensor of the one or more objects entering the cavity; and
a lighting apparatus disposed on an outer surface of the garment in operative communication with the disinfecting device, the lighting apparatus comprising:
a clean visual indicator,
an unclean visual indicator, and
a switch configured to:
responsive to a predetermined amount of time passing and the disinfecting portion not being actuated, display the unclean visual indicator and stop display of the clean visual indicator, and
responsive to the disinfecting portion being activated, display the clean visual indicator and stop display of the unclean visual indicator.

2. The garment of claim 1, wherein the sensor is a reed switch.

3. The garment of claim 1, wherein the sensor is a motion sensor.

4. The garment of claim 1, wherein the sensor is coupled to the switch.

5. The garment of claim 1, further comprising a vibrational element configured to vibrate at least a portion of the garment.

6. The garment of claim 5, wherein the vibrational element is configured to vibrate during actuation of the disinfecting portion.

7. The garment of claim 1, wherein the disinfecting portion comprises a light emitting diode (LED).

8. The garment of claim 7, wherein the LED is operative to emit light effective to disinfect when shone on a surface.

9. The garment of claim 7, wherein the LED is arranged on a first portion of the cavity and a second LED is arranged on a second portion of the cavity that opposes the first portion of the inner cavity.

10. The garment of claim 1, wherein the cavity has an opening comprising a first flexible portion and a second flexible portion, the first flexible portion being configured to releasably secure to the second flexible portion.

11. The garment of claim 10, wherein the sensor is connected to the first flexible portion and the second flexible portion.

12. A disinfecting system comprising:
a disinfecting device comprising:
a dispensing portion configured to administer a predetermined amount of sanitizing fluid, and
a first proximity sensor coupled to the disinfecting portion and operative to transmit a signal responsive to the following steps being performed:
a second proximity sensor being within a predetermined distance of the first proximity sensor, and
administration of the predetermined amount of sanitizing fluid; and
the lighting apparatus disposed on an outer surface of a garment of a user and in operative communication with the disinfecting device, the lighting apparatus comprising:
a display portion configured to visually display one of a clean visual indicator and an unclean visual indicator, and
a switch coupled to the second proximity sensor, the switch configured to:
responsive to a predetermined amount of time passing and the disinfecting portion not being actuated, display the unclean visual indicator on the display portion, and
responsive to receiving the signal transmitted from the first proximity sensor, display the clean visual indicator on the display portion.

13. The disinfecting system of claim 12, wherein the disinfecting device is configured to secure to a garment of a user.

14. The disinfecting system of claim 12, wherein the predetermined proximity is adjustable.

15. A disinfecting system comprising:
a disinfecting device comprising:
a cavity,
a bacteria detection sensor operative to:
actuate upon one or more objects entering the cavity, and
detect a bacterial amount on the object,
a disinfecting portion configured to disinfect the one or more objects within the cavity, the disinfecting portion being actuated upon receiving an indication from the sensor of the one or more objects entering the cavity; and
a lighting apparatus disposed on an outer surface of a garment in operative communication with the disinfecting device, the lighting apparatus comprising:
a display portion, and
a switch configured to:
responsive to a predetermined amount of time passing and the disinfecting portion not being actuated, display the unclean visual indicator on the display portion, and
responsive to the disinfecting portion being activated and the bacteria detected on the object, via the bacteria detection sensor, falling below a predetermined threshold, display the clean visual indicator on the display portion.

16. The disinfecting system of claim 15, wherein the disinfecting device and the lighting apparatus are configured to be secured to a garment of a user.

17. The disinfecting system of claim 15, wherein the disinfecting portion comprises a light emitting diode (LED).

18. The disinfecting system of claim 15, further comprising a vibrational element configured to vibrate at least a portion of the garment during actuation of the disinfecting portion.

19. The disinfecting system of claim 15, wherein the cavity comprises an opening having a first flexible portion and a second flexible portion, the first flexible portion being configured to releasably secure to the second flexible portion.

20. The disinfecting system of claim 19, wherein a reed switch is connected to the first flexible portion and the second flexible portion operative to actuate the bacteria detection responsive to the first flexible portion being separated from the second flexible portion.
